(19) United States
(12) Patent Application Publication
(10) Pub. No.: US 2007/0100300 A1
(43) Pub. Date: May 3, 2007

(54) Ear fluid removal device

(76) Inventor: Shohreh Hamed Hashemian, Knoxville, TN (US)

Correspondence Address: KNOX PATENTS, P.O. BOX 30034, KNOXVILLE, TN, 37930-0034, US

(21) Appl. No.: 11/264,978

(22) Filed: Nov. 2, 2005

(51) Int. Cl. A61M 31/00; A61M 37/00

(52) U.S. Cl. 604/275; 604/133

(57) ABSTRACT

An ear fluid removal device for manually removing fluid and debris from the ear canal. The ear fluid removal device is a tool for removing fluid, particulates, or other debris from the ear canal while reducing the possibility of injury to the ear canal or surrounding tissues. The ear fluid removal device includes an elongated flexible tube attached to an earpiece such that, when suction is applied to the tube end opposite the earpiece, the earpiece is capable of aspirating fluid and debris from the ear canal of the user and transferring the fluid and debris into the flexible tube. The flexible tube is of a length to span the distance between the mouth of the user and the user's ear. In one embodiment, the tube has a length sufficient to form a loop for the collection of fluid and debris.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention pertains to a device for removing fluid accumulated in a person's ear canal. More particularly, this invention pertains to a device that is operated by a person, who provides suction power, to control the removal of fluid that has entered the ear canal, such as by swimming or bathing.

2. Description of the Related Art

The ear canal is prone to collection of water after swimming, diving, surfing, and the like. Accumulation of water within the ear and development of infections resulting from ear-water accumulation are common. Non-mechanical hand held curettes have been the traditional method of wax removal by physicians. Fluid removal is typically performed in a physician's office by using large electrical powered vacuum pumps connected to tubing and to narrow gauge tips under direct visual observation to evacuate fluid and debris. Cotton tipped swabs are often used inappropriately in the ear canal to remove wax and fluid, however, such use can cause trauma to the tissues of the ear as well as worsen the impaction of fluid and debris.

Numerous devices have been proposed and implemented for extracting fluids, particulates, and other matter from different orifices and tissues of human and animal bodies. Among them, U. S. Pat. No. 69,110, issued to Morris Mattson on Sep. 24, 1867, discloses a flexible tube interposed between an elastic bulb and a discharge pipe. The elastic bulb provides suction to the discharge pipe in order to take up and discharge fluids.

U. S. Pat. No. 4,334,538, issued to Steven K. Juhn on Jun. 15, 1982, discloses an aspirator for manually collecting liquid samples. The '538 device comprises a handle connected to a vacuum source. The handle has a sample collection vial fitting into a cavity in the handle with a finger control vent in the cavity. A tube leads into the sample collection vial and functions as the probe for the sample to be collected.

U. S. Pat. No. 5,062,835, issued to Carlos A. Maitz on Nov. 5, 1991, discloses an aspirator device for removing body fluids, comprising a fluid collector with an air-type chamber attached to a flexible tube to be inserted in a body cavity. The fluid collector is connected to a manually operable vacuum means or squeeze bulb. The manually operable vacuum means is operatively associated with first and second one-way valves establishing a vacuum for removing and depositing bodily fluids in the fluid collector.

U. S. Pat. No. 5,665,094, issued to Robert Arlin Goldenberg on Sep. 9, 1997, discloses an apparatus for collecting a middle ear fluid specimen through the ear drum of a patient. The '094 device utilizes a hollow needle mountable on a bulb. The bulb is operatively adapted to draw a specimen of middle ear fluid into the hollow needle when the needle is mounted on the bulb.

U. S. Pat. No. 6,059,803, issued to Daniel A. Spilman on May 9, 2000, discloses a portable hand held ear vacuum device for removing fluid and debris from an ear canal. The '803 device includes a handle with a motor linked to a fan and a control switch. A collection chamber for collecting fluid and other debris from an ear canal is secured to the handle. The collection chamber has a reservoir for collecting and storing accumulated fluid and debris from the ear canal. An ear insertion element is secured to the collection chamber. The ear insertion element has an aperture continuous with the reservoir in the collection chamber. The motor in the handle creates a vacuum within the collection chamber, enabling fluids and debris to be drawn from the ear canal into the reservoir by suction.

### BRIEF SUMMARY OF THE INVENTION

An ear fluid removal device for manually removing fluid and debris from the ear canal is disclosed. The ear fluid removal device provides a tool for removing fluid, particulates, or other debris from the ear canal while reducing the possibility of injury to the ear canal or surrounding tissues.

The ear fluid removal device includes an elongated flexible tube attached to an earpiece. The tube has a first end and a second end. The first end is adapted to be received by the mouth of a user. The second end is secured to the earpiece. The flexible tube is of sufficient length to span the distance between the mouth of the user and the user's ear. In one embodiment, the flexible tube is at least translucent and is of sufficient length as to allow the user to visually observe and monitor the aspiration and extraction of fluid and debris from the ear canal of the user.

In one embodiment, the earpiece is a piece of resiliently deformable material adaptable to be received by the ear of a user. The deformability of the earpiece allows the earpiece to compress for insertion and expand to substantially fill and conform to the contour of the user's ear canal. In one embodiment, the earpiece is a single piece of fluid-permeable material, such as open-cell foam. The earpiece connects to the tube second end such that the earpiece substantially surrounds and encloses the tube second end.

Another embodiment provides an earpiece having a through-opening, the through-opening receiving the flexible tube second end. In this embodiment, the earpiece is a piece of substantially fluid-impermeable material, such as closed-cell foam. In still another embodiment, the earpiece includes a piece of resiliently deformable material, such as open or closed-cell foam, substantially covered by a fluid-impermeable membrane.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a side view of one embodiment of an ear fluid removal device, showing the ear piece inserted into the ear of a user with the user providing suction to the tube;

FIG. 2 is a perspective view of the ear fluid removal device of FIG. 1, showing the flexible tube and the earpiece;

FIG. 3A is a cross-sectional side view of one embodiment of the ear fluid removal device; and

FIG. 3B is a cross-sectional side view showing another embodiment of the ear fluid removal device.

### DETAILED DESCRIPTION OF THE INVENTION

An ear fluid removal device for manually removing fluid and debris from the ear canal is disclosed. The ear fluid removal device provides a tool for removing fluid, particulates or other debris from the ear canal while reducing the possibility of injury to the ear canal or surrounding tissues. The ear fluid removal device allows the user to control the specific level of suction applied to the ear canal, and the device is usable by most individuals without specific medical training.

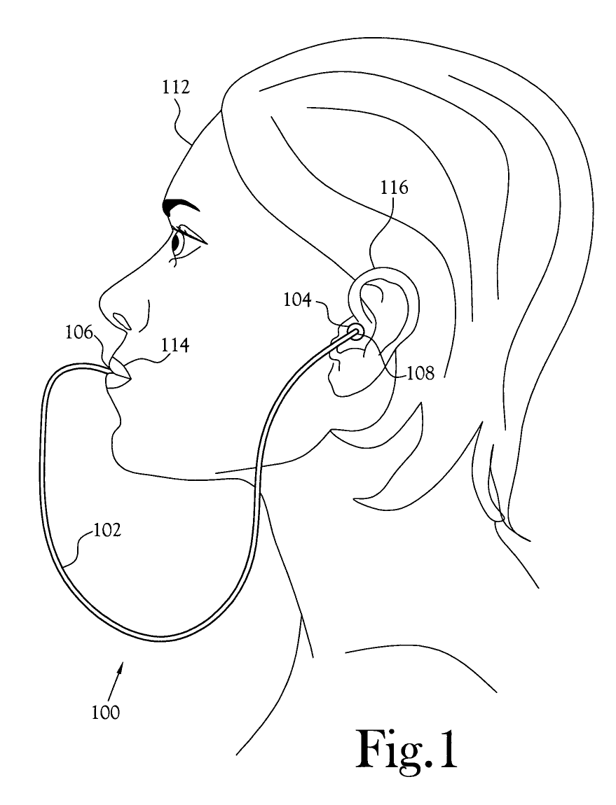

FIG. 1 illustrates a side elevation view of one embodiment of an ear fluid removal device 100. As shown in FIG. 1, the ear fluid removal device 100 includes an elongated flexible tube 102 attached to an earpiece 104. The tube 102 is a conduit with a first end 106 and a second end 108. The first end 106 is adapted to be received by the mouth 114 of a user 112. The second end 108 is secured to the earpiece 104, in one embodiment, by a frictional connection achieved by inserting the second end 108 of the tube 102 into an opening in the 5 earpiece 104. In other embodiments, the second end 108 is secured to the earpiece 104 by mechanical fastening means, welds, adhesives, or by providing an elongated flexible tube 102 and an earpiece 104 as an integral unit. In still another embodiment, the second end 108 is detachably secured to the earpiece 104.

The flexible tube 102 is of sufficient length to span the distance between the mouth 114 of the user 112 and the user's ear 116. In one embodiment, the flexible tube 102 is of sufficient length as to allow the user 112 to visually observe the flexible tube 102 while the earpiece 104 is inserted into the user's ear 116 and the tube first end 106 is inserted into the user's mouth 114. Additionally, at least a portion of the flexible tube 102 is translucent, such that the user 112 is able to determine the presence of fluid and debris inside the tube 102. The translucent tube 102 allows the user 112 to visually monitor the aspiration and extraction of fluid and debris from the ear 116. This feature allows the user to monitor collection of fluid and debris within the flexible tube 102, so as to avoid inhalation of fluid and debris removed from the ear 116. In another embodiment, the tube 102 is of such a length that a portion of the tube 102 serves as a reservoir for the collection of any fluid and/or debris collected from the ear 116, thereby preventing the inhalation of any fluid or debris by the user 116. Such a length would be sufficient if the tube 102 forms at least one loop between the two ends 106, 108 when the device 100 is in use.

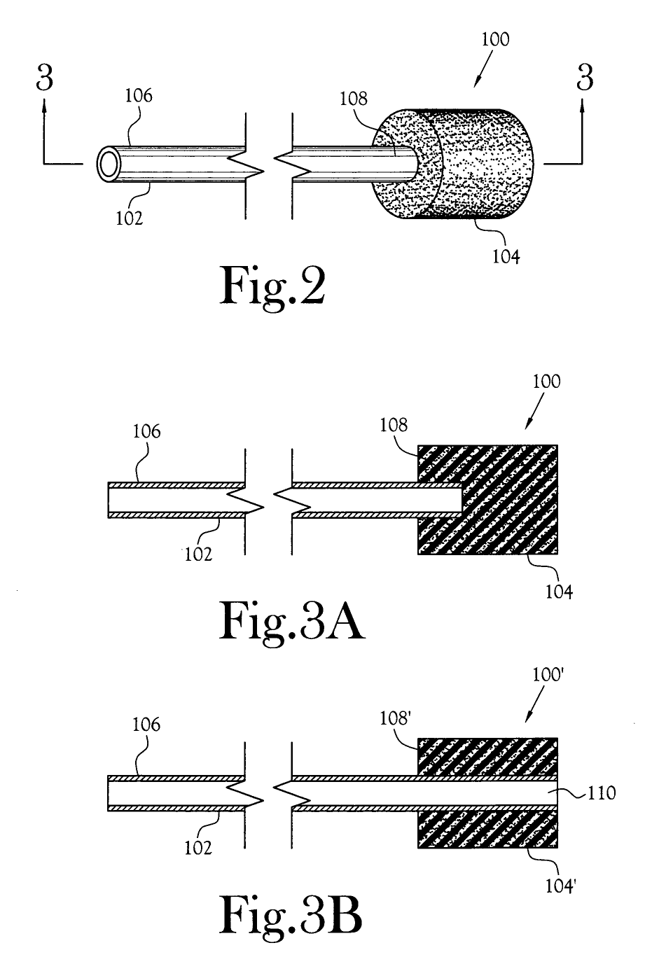

FIG. 2 is a perspective view of the ear fluid removal device 100 of FIG. 1. As shown in FIG. 2, the earpiece 104 is a piece of resiliently deformable material adaptable to be received by the ear 116 of a user 112. The deformability of the earpiece 104 allows the earpiece 104 to be compressed for insertion and to subsequently expand to substantially fill and conform to the contour of the user's outer ear canal. The second end 108 of the tube 102 is connected to the earpiece 104 such that, when suction is applied to the tube first end 106, a vacuum is created within the tube 102 and transferred to the earpiece 104. When actuated by the vacuum, the earpiece 104 is capable of collecting fluid within the ear and transferring the fluid into the elongated flexible tube 102.

FIG. 3A is a cross-sectional view of the ear fluid removal device. As illustrated in FIG. 3A, the earpiece 104 includes a single piece of fluid-permeable material. In one embodiment, the earpiece 104 is made of an open-cell foam. The earpiece 104 connects to the tube second end 108 and the earpiece 104 substantially surrounds and encloses the tube second end 108. Those skilled in the art will recognize many other materials are suitable for use in the earpiece.

In use, the earpiece 104 is inserted into the ear 116 of the user 112, and the tube first end 106 is inserted into the user's mouth 114. The earpiece 104 absorbs fluid and particulates within the ear canal. The flexible tube 102 serves as a storage reservoir for the removed fluid and debris. The user 112 applies suction to the tube 102 by sucking on the tube first end 106 with the user's mouth 114. The suction provided by the user 112 creates negative pressure within the flexible tube 102, which in turn creates negative pressure within the pores of the earpiece 104. This vacuum within the earpiece 104 helps to increase the absorption of fluid by the earpiece 104 within the ear canal. The user 112 views the flexible tube 102 to monitor the aspiration and collection of fluid and debris from the ear 116. In another embodiment, the tube 102 is of sufficient length that it serves as a reservoir for the collection of any fluid and/or debris collected from the ear 116. After fluid and debris is absorbed by the earpiece 104, the user 112 removes the earpiece 104 from the ear 116. The earpiece 104 and the flexible tube 102 are then cleaned, and the fluid and debris is discarded. Alternatively, after removal from the ear 116, the earpiece 104 itself is discarded and replaced.

FIG. 3B shows a cross-sectional view of another embodiment of the ear fluid removal device 100 of the present invention. In the illustrated embodiment, the earpiece 104′ includes a through-opening 110 that receives the flexible tube second end 108′. The earpiece 104′ surrounds the end 108 of the tube 102, and the tube 102 completely penetrates the earpiece 104′. When inserted into the ear 116, the earpiece 104′ expands to substantially fill and conform to the contour of the user's ear canal. When the user 112 provides suction to the tube 102′, fluid and particulates are collected through the through-opening 110 and deposited into the tube 102 through the tube second end 108. The amount of vacuum created in the ear canal by the user 112 is limited by air entry from outside the ear 116 through the earpiece 104′. Therefore, the user 112 provides suction that causes air flow from outside the ear 116 and that air flow also carries the fluid and debris from inside the ear canal.

In the illustrated embodiment, the earpiece 104′ includes a piece of substantially fluid-impermeable material. In one embodiment, the earpiece 104′is made of an closed-cell foam. In another embodiment, the earpiece 104′includes a piece of resiliently deformable material with the cylindrical outer portion substantially covered by a fluid-impermeable membrane and the flat end inserted into the ear 116 adapted to absorb and pass fluids. That is, the cylindrical face of the earpiece 104 has a fluid-impermeable surface.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described.

Accordingly, departures may be made from such details without departing from the spirit or scope of applicant's general inventive concept.

What is claimed is:

1. An apparatus for manually aspirating and removing fluid and debris from an ear canal of a user, said apparatus comprising:
a conduit having a first end and a second end opposite said first end, said conduit being a tubular member defining a passage between said first end and said second end, said conduit being flexible, said conduit being translucent, said first end adapted to be received by a mouth of the user, said conduit having a length wherein a portion of said conduit is visible to the user when said apparatus is in use; and
an earpiece secured to said conduit proximate said second end, said earpiece having a generally cylindrical shape, said earpiece being resiliently deformable wherein said earpiece is adapted to be received by the ear canal when said earpiece is compressed, said earpiece adapted to conform to the ear canal after said earpiece is compressed and received by the ear canal, said earpiece being permeable to fluid and air;
whereby suction applied by a user transfers the fluid and debris from the ear canal to the earpiece.

2. The apparatus of claim 1 wherein said earpiece substantially surrounds and encloses said second end of said conduit.

3. The apparatus of claim 1 wherein said earpiece substantially surrounds said second end of said conduit and said second end penetrates said earpiece.

4. The apparatus of claim 1 wherein said earpiece includes a cylindrical section of open-cell foam.

5. The apparatus of claim 1 wherein said earpiece includes a cylindrical section of open-cell foam, and a cylindrical face of said cylindrical section of said earpiece has a fluid impermeable surface.

6. An apparatus for manually aspirating and removing fluid and debris from the ear canal of a user, said apparatus comprising:
a member being a elongated flexible tube defining a conduit through which air can pass between a first end and a second end of said member, said first end of said member adapted to be received by the user's mouth; and
an earpiece secured to said member proximate said second end, said earpiece including a resiliently deformable material adapted to allow said earpiece to deform for insertion into the user's ear canal and to expand to substantially fill the user's ear canal, said earpiece adapted to absorb fluid and convey the absorbed fluid to said second end of said member, wherein
said earpiece secured to said second end such as to substantially surround said member second end.

7. The apparatus of claim 6 wherein said member includes a translucent section, and said member has a length wherein the user is able to visually observe said translucent section of said member when said earpiece is inserted into the user's ear and said first end of said member is inserted into the user's mouth.

8. The apparatus of claim 6 wherein said member has a length wherein said member includes at least one loop between said first end and said second end when said first end is received by the user's mouth and said earpiece is received by the ear canal.

9. The apparatus of claim 6 wherein said earpiece has a generally cylindrical shape.

10. The apparatus of claim 6 wherein said earpiece substantially surrounds and encloses said second end of said conduit.

11. The apparatus of claim 6 wherein said earpiece substantially surrounds said second end of said conduit and said second end penetrates said earpiece.

12. The apparatus of claim 6 wherein said earpiece includes a cylindrical section of open-cell foam.

13. The apparatus of claim 6 wherein said earpiece includes a cylindrical section of open-cell foam, and a cylindrical face of said cylindrical section of said earpiece has a fluid impermeable surface.

14. A method for manually aspirating and removing fluid and debris from the ear canal, said method comprising the steps of:
a) compressing an earpiece formed of a resiliently deformable material, said earpiece connected to a conduit having a first end opposite said earpiece, said conduit being a tubular member defining a passage between said first end and said earpiece;
b) inserting said earpiece into the ear canal of a user, wherein said earpiece expands to conform to a contour within the user's ear canal;
c) inserting said first end of said conduit into the mouth of the user; and
d) applying oral suction to said first end of said conduit, thereby causing said earpiece to draw fluid and debris from the ear canal of the user.

15. The method of claim 14 wherein said step d) of applying oral suction includes the steps of visually observing a translucent portion of said conduit and stopping oral suction when fluid or debris is observed in said translucent portion.
